(19) United States
(12) Patent Application Publication
(10) Pub. No.: US 2014/0207012 A1
(43) Pub. Date: Jul. 24, 2014

(54) SYSTEMS AND METHODS FOR ANALYZING ELECTROCARDIOGRAMS TO DETECT VENTRICULAR FIBRILLATION

(71) Applicants: Jason Coult, Seattle, WA (US); Christopher Neils, Lake Forest Park, WA (US); Mickey Eisenberg, Seatac, WA (US); Thomas Rea, Seattle, WA (US); Peter J. Kudenchuk, Normandy Park, WA (US); Lawrence Duane Sherman, Bellevue, WA (US)

(72) Inventors: Jason Coult, Seattle, WA (US); Christopher Neils, Lake Forest Park, WA (US); Mickey Eisenberg, Seatac, WA (US); Thomas Rea, Seattle, WA (US); Peter J. Kudenchuk, Normandy Park, WA (US); Lawrence Duane Sherman, Bellevue, WA (US)

(73) Assignee: Univeristy of Washington through its Center for Commercialization, Seattle, WA (US)

(21) Appl. No.: 14/126,411

(22) PCT Filed: Jul. 2, 2012
(86) PCT No.: PCT/US12/45292

§ 371 Date: Mar. 27, 2014

Related U.S. Application Data
(60) Provisional application No. 61/503,444, filed on Jun. 30, 2011.

(51) Int. Cl. A61B 5/046; A61B 5/0452

(52) U.S. Cl. CPC A61B 5/046; A61B 5/04525
USPC 600/518; 600/515

(57) ABSTRACT

The present technology describes various embodiments of systems and methods for analyzing electrocardiograms to detect ventricular fibrillation. In several embodiments, systems for detecting ventricular fibrillation can be implemented without interrupting cardiopulmonary resuscitation. In one embodiment, a method of identifying a cardiac rhythm in a person includes recording an electrocardiogram signal of the person and stratifying the signal. A signal having a parameter value within a pre-determined range is categorized as a shockable ventricular fibrillation signal while a signal having a parameter value outside the pre-determined range is categorized as a non-shockable signal.

### CROSS-REFERENCE TO RELATED APPLICATION

This application is a U.S. national phase of PCT/US2012/045292, filed on Jul. 2, 2012, which claims the benefit of U.S. Provisional Patent Application No. 61/503,444, filed Jun. 30, 2011, all of which are incorporated herein by reference in their entirety.

### TECHNICAL FIELD

The present technology is generally directed to systems and methods for analyzing electrocardiograms. More specifically, some embodiments are directed to systems for detecting ventricular fibrillation without interrupting cardiopulmonary resuscitation.

### BACKGROUND

Approximately a third of the 300,000 cases of cardiac arrest each year in the United States are due to ventricular fibrillation (VF). The only means to terminate this lethal arrhythmia is by the timely delivery of an electric shock to defibrillate the heart. In order to determine whether VF is the cause of any given patient's collapse, it is necessary to connect a cardiac monitor to the patient by means of at least two electrodes which may then carry the signal to a device which can amplify and analyze the signal to determine if the rhythm present is VF, asystole, or an organized cardiac rhythm.

Such analytic devices can be bulky and cumbersome, and can be susceptible to monitoring noise. For example, many devices are unable to distinguish between VF and chest compressions from cardiopulmonary resuscitation (CPR) efforts. However, requiring that CPR be stopped to test for ventricular fibrillation can have an immediate adverse effect on the patient. Further, monitoring noise and artifacts can be prevalent in noisy hospitals and moving ambulances, triggering false alarms by the monitoring devices. Medical professionals may fall into the habit of ignoring these false alarms, while the patient may be in real distress.

### BRIEF DESCRIPTION OF THE DRAWINGS

FIG. 1 is a partially schematic illustration of an electrocardiogram system configured in accordance with embodiments of the technology.

FIG. 2 is a block diagram illustrating a method of analyzing an electrocardiogram signal to detect ventricular fibrillation configured in accordance with embodiments of the technology.

FIG. 3A is a block diagram illustrating a decision tree implemented by a rhythm solver function for an automatic external defibrillator configured in accordance with embodiments of the technology.

FIG. 3B is a block diagram illustrating a decision tree implemented by a rhythm solver function for another automatic external defibrillator model configured in accordance with embodiments of the technology.

FIG. 4 is a block diagram illustrating a method of determining a Cross-correlation Low Amplitude Sum of an electrocardiogram signal in accordance with embodiments of the technology.

FIG. 5 is a block diagram illustrating a method of determining a Fixed Low Amplitude Threshold Sum of an electrocardiogram signal in accordance with embodiments of the technology.

### DETAILED DESCRIPTION

The present technology describes various embodiments of systems and methods for analyzing electrocardiograms to detect ventricular fibrillation. In several embodiments, systems for detecting ventricular fibrillation can be implemented without interrupting cardiopulmonary resuscitation. In one embodiment, for example, a method of identifying a cardiac rhythm in a person includes recording an electrocardiogram signal of the person and stratifying the signal. A signal having a parameter value within a pre-determined range is categorized as a shockable ventricular fibrillation signal while a signal having a parameter value outside the pre-determined range is categorized as a non-shockable signal.

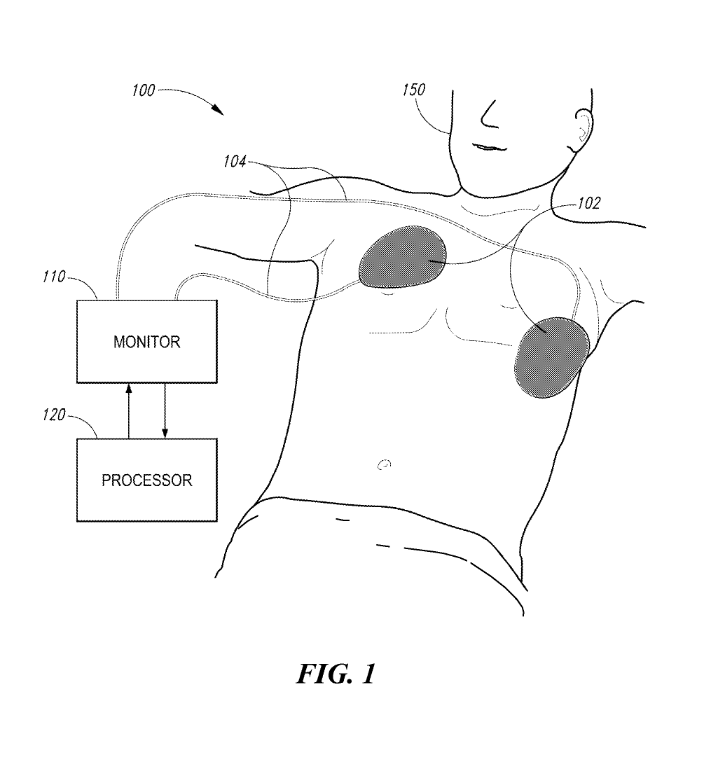

Specific details of several embodiments of the technology are described below with reference to FIGS. 1-5. Other details describing well-known structures and systems often associated with electrocardiograms have not been set forth in the following disclosure to avoid unnecessarily obscuring the description of the various embodiments of the technology. Many of the details, dimensions, angles, and other features shown in the Figures are merely illustrative of particular embodiments of the technology. Accordingly, other embodiments can have other details, dimensions, angles, and features without departing from the spirit or scope of the present technology. A person of ordinary skill in the art, therefore, will accordingly understand that the technology may have other embodiments with additional elements, or the technology may have other embodiments without several of the features shown and described below with reference to FIGS. 1-5.

FIG. 1 is a partially schematic illustration of an electrocardiogram (ECG) system 100 (“the system 100”) configured in accordance with embodiments of the technology. The system 100 includes a cardiac monitor 110 coupled to a patient 150 by means of two or more electrodes 102 coupled to the monitor 110 with wires 104. The electrodes 102 may be monitoring a voltage signal, impedance, and/or other values. The monitor 110 may then carry the signal to a device (e.g., a processor 120) which can amplify and analyze the signal. In further embodiments, the monitor 110 and processor 120 may comprise a single, unitary device, or may comprise more than two devices.

The processor 120 can be part of any personal computer, tablet, mobile computing device, or other physical computer-readable storage medium. The processor 120 can be configured to implement instructions which comprise a method to analyze the time series voltages of an ECG as derived from the patient 150 and to determine if the rhythm is shockable (VF) or non shockable (asystole or an organized rhythm). In several embodiments, this determination can be made either while the patient 150 is lying undisturbed or during active cardiopulmonary resuscitation (CPR). This allows for a reliable determination of the cardiac rhythm without stopping therapy. In some embodiments, the processor 120 provides a determination of whether a patient in VF would respond to an electric shock with an organized rhythm. This predictive ability allows other therapies to be provided to patients 150 who have little or no probability of responding to a shock. It also allows identification of those patients 150 with a very high probability of achieving an organized rhythm after electric shock so that treatment could be delivered immediately. In some embodiments, the system 100 will automatically implement a shock to the patient 150 if it determines such a treatment would be beneficial. In further embodiments, the system 100 alerts a user to implement such a treatment to the patient 150. In various embodiments, the system 100 can be sized for personal use (e.g., within the home), for use at a hospital, for use by first responders, or for other locations or uses. In still further embodiments, the system 100 can be incorporated into a larger monitoring or treatment system. The system 100 can include features generally similar to automatic external defibrillators (AED) known in the art.

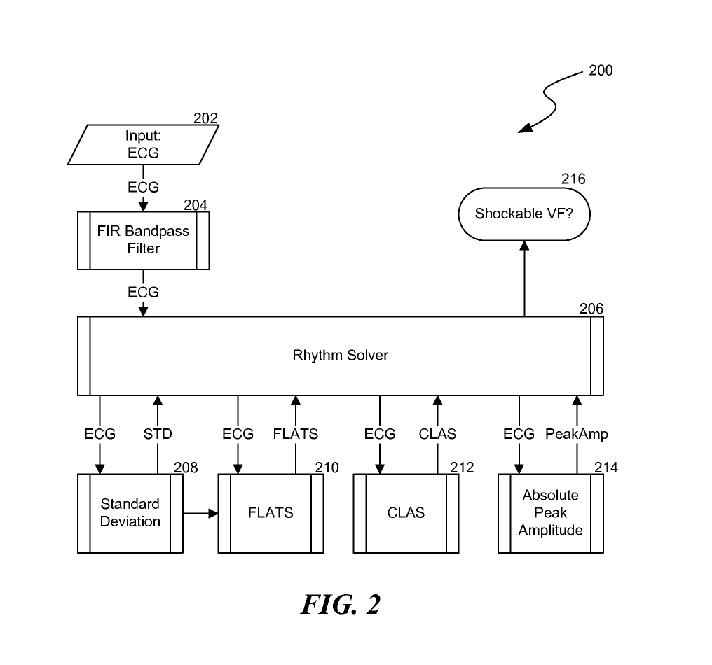

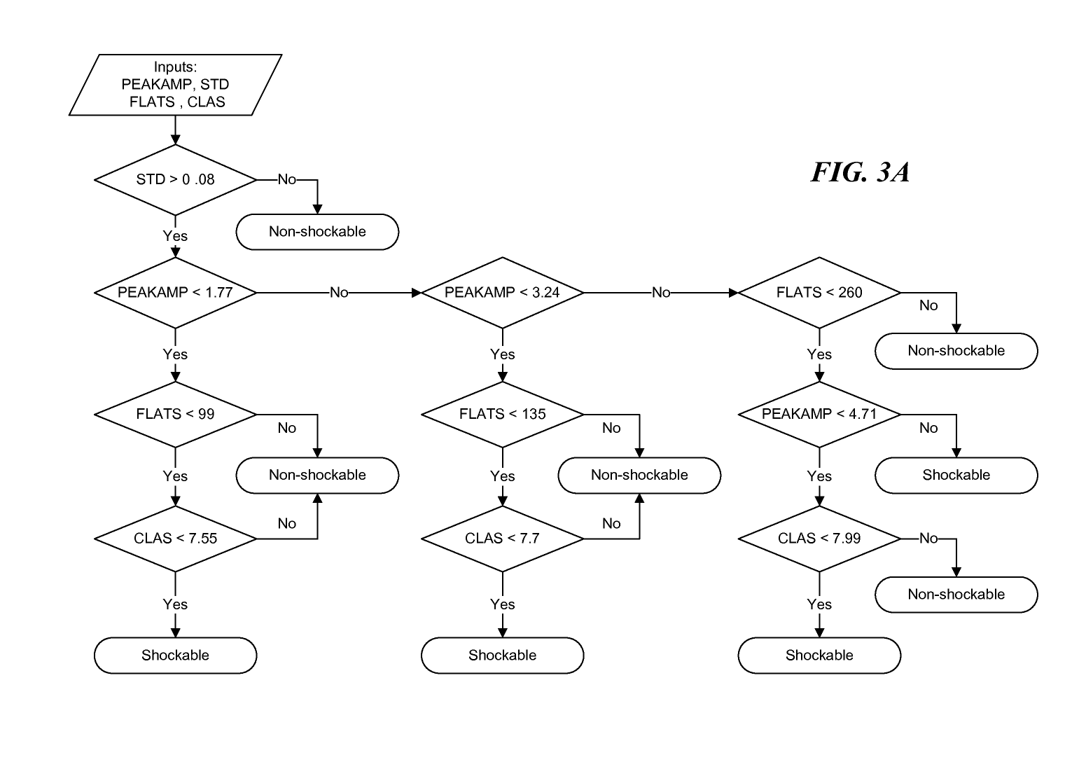

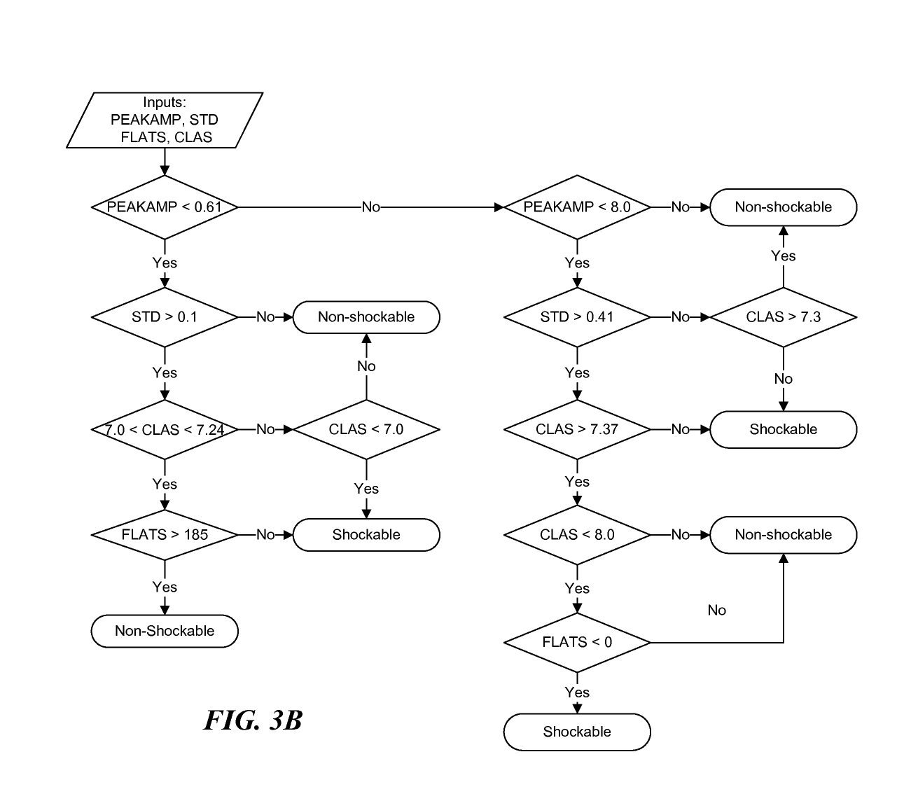

FIG. 2 is a block diagram illustrating a method 200 of analyzing an ECG to detect VF in accordance with embodiments of the technology. More details regarding several aspects of this method 200 are provided below with reference to FIGS. 3A-5. At block 202, the method 200 includes, for example, inputting an ECG signal into a processor (e.g., the processor 120). The ECG signal is recorded by the processor 120 from the patient's chest using a monitor such as the monitor 110 described above. In some embodiments, the monitor 110 amplifies and performs an analog to digital conversion of the signal prior to providing it to the processor 120. The signal can be sampled at over 100 samples per second and in an amplitude range of from −10 to 10 mV. In some embodiments, the signal is converted from analog to digital at 250 samples per second.

At block 204, the ECG signal can be band-pass filtered to remove noise and/or artifacts. Depending on the specific model of AED being used, it may or may not be necessary to filter the input ECG signal. In particular embodiments, the ECG signal is filtered with a band-pass, equiripple, 54-tap FIR filter to remove baseline drift and high frequency noise. Depending on the AED, the specific filter characteristics may change. The filter can be designed in such a way as to only preserve frequencies that need to be analyzed by the algorithm, and implemented so that the filter artifact is distributed to either end of the ECG clip being analyzed, as opposed to distributed to only one end of the clip. In further embodiments, other filters or filtering techniques can be used.

The method 200 further includes at block 206 implementing a “Rhythm Solver” function to the signal to determine whether the signal indicates shockable VF. The Rhythm Solver function comprises a decision tree that implements a series of transformations and calculations (blocks 208, 210, 212, and 214) to analyze the signal. These calculations may be applied individually or in combination to provide the classification. In several embodiments, the length of recording used to perform the calculations is less than 10 seconds. In one embodiment a 3.8 second recording at 250 samples per second is used. It may be possible to make the determination in a shorter time period with 1 or 2 seconds of signal acquisition being sufficient in some cases.

At block 216, the Rhythm Solver decision tree stratifies the signal; signals with parameter values within a certain range are classified as shockable, while signals with parameter values outside the range are classified as non-shockable. The cutoff values, type of parameters, and/or order by which the parameters are used to classify the rhythm can vary depending on the AED, cardiac monitor, or other device from which the ECG signals are obtained. Data obtained from one type of AED or other monitoring device will require different thresholds and decision sequences than data obtained from a different device. Frequency response, filtering characteristics, preamp gain, and other factors that vary between device models necessitate the tuning of the Rhythm Solver to different machines. Rhythm Solver functions tuned for two different representative device model types are discussed below with reference to FIGS. 3A and 3B.

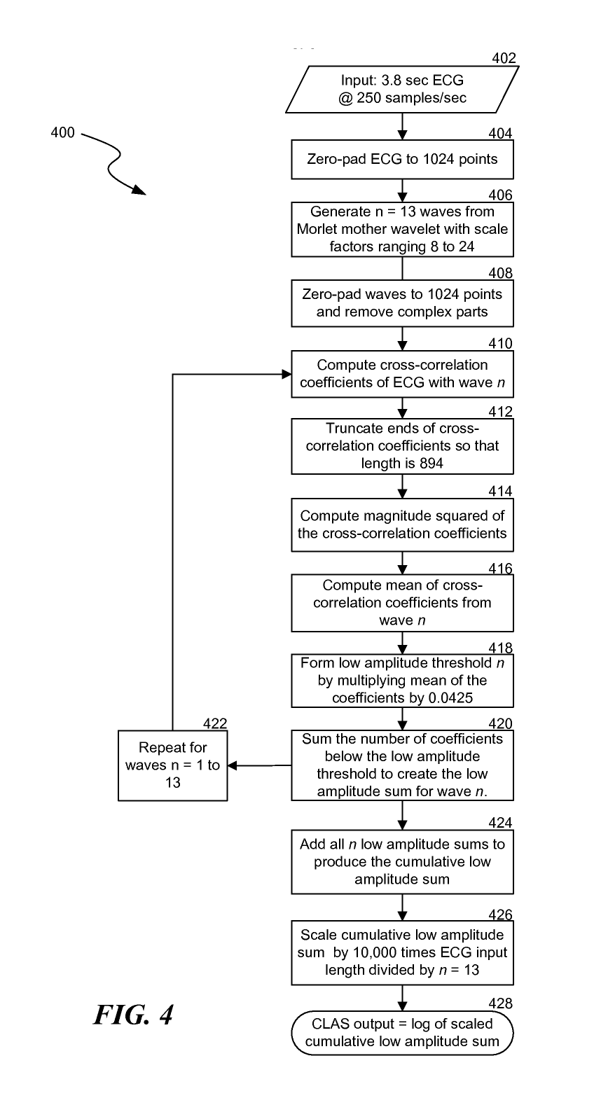

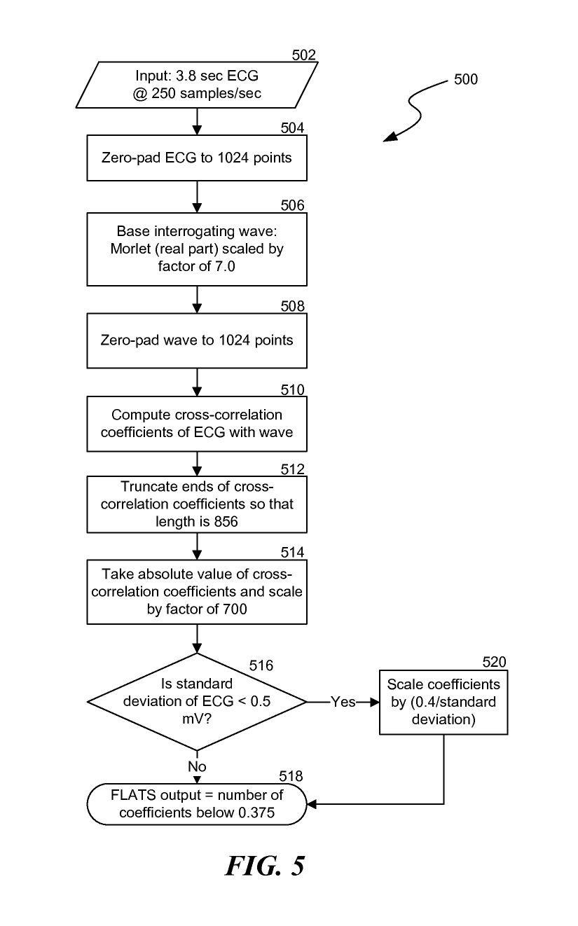

The Rhythm Solver function uses parameters labeled as standard deviation (STD), Fixed Low Amplitude Threshold Sum (FLATS), Cross-correlation Low Amplitude Sum (CLAS), and Absolute Peak Amplitude (PEAKAMP) (blocks 208-214) to classify the input ECG signal. In further embodiments, more or fewer parameters may be used. The STD is simply the standard deviation of the signal. The PEAKAMP is the maximum of the absolute value of the ECG signal. The CLAS and FLATS are calculated values that can be used in combination to distinguish shockable VF from non-shockable asystole and organized rhythms. In some embodiments, the CLAS can be useful to distinguish VF from Organized rhythms during CPR, while the FLATS can be useful to distinguish VF from asystole during CPR. The CLAS will be discussed in further detail below with reference to FIG. 4 and the FLATS will be discussed in further detail below with reference to FIG. 5.

FIG. 3A is a block diagram illustrating a decision tree implemented by a rhythm solver function for a first AED model in accordance with embodiments of the technology. FIG. 3B is a block diagram illustrating a decision tree implemented by a rhythm solver function for another AED model in accordance with embodiments of the technology. Referring to FIGS. 3A and 3B together, these functions can be developed by observing the parameter values for ECG training data, sorting the data, and applying cutoffs for optimal and/or preferred separation of shockable and non-shockable rhythm values. In further embodiments, some or all of the STD, PEAKAMP, FLATS, CLAS, or other parameters may have different cut-off values or determinative ranges.

FIG. 4 is a block diagram illustrating a method 400 of determining a CLAS of an ECG signal. At block 402, the method 400 includes inputting an ECG signal into a processor. In a particular embodiment, the signal comprises a 3.8 second signal, where each second is divided into 250 sample points, leading to a total sample group of 950 points. Other signal durations or sample sizes may be used in further embodiments. At block 404, zeros are added to the beginning and end of the sample group to create a sample group of 1024 points. In further embodiments, this step may be omitted or the sample group may have other sizes.

At block 406, an interrogation wave is used to interrogate the unknown rhythm epoch. In some embodiments, a Morlet mother wavelet is used and generates a plurality (e.g., 13) of waves scaled by various factors (e.g., 8 to 24). In further embodiments, other waves can be used. At block 408, the waves are again zero-padded to create a sample group having 1024 points. In some embodiments, complex parts of the waves are removed.

At block 410, cross-correlation coefficients of the ECG signal are computed for an individual wave. At block 412, the ends of the cross-correlation can be truncated to a pre-selected length (e.g., 894 sample points). The cross-correlation coefficients are squared (block 414) and a mean value of the squared coefficients is calculated (block 416). At block 418, a low amplitude threshold is generated by scaling the mean by a factor (e.g., by 0.0425). This factor represents a percentage of the mean below which coefficients will be counted. At block 420, the number of coefficients below the low amplitude threshold is summed to create the low amplitude sum for the individual wave. At block 422, this process is repeated for each individual wave.

At block 424, the low amplitude sums for each individual wave are summed to produce the cumulative low amplitude sum. At block 426, this value can be scaled by a factor (e.g., 10,000 multiplied by the signal length and divided by the number of waves, such as 13). At block 428, the log can be taken of the scaled cumulative low amplitude sum, with the result set as the CLAS value. As discussed above, the CLAS value can be particularly useful in distinguishing a VF rhythm from an organized rhythm.

FIG. 5 is a block diagram illustrating a method 500 of determining a FLATS value of an ECG signal. The FLATS is similar to the CLAS in that it uses the cross-correlation of a wave with the ECG, but is tailored more to discriminate non-shockable asystole from VF, especially during CPR. At block 502, the method 500 includes inputting an ECG signal into a processor. In a particular embodiment, the signal comprises a 3.8 second signal, where each second is divided into 250 sample points, leading to a total sample group of 950 points. Other signal durations or sample sizes may be used in further embodiments. At block 504, zeros are added to the beginning and end of the sample group to create a sample group of 1024 points. In further embodiments, this step may be omitted or larger or smaller sample groups may be used. At block 506, a base interrogating wave is selected and can be scaled. In a particular embodiment, the wave comprises the real portion of a complex Morlet mother wavelet, and the scale factor is 7.0. At block 508, the wave is again zero-padded to comprise a group sample of 1024 points. Again, other group sample sizes can be used.

At block 510, cross-correlation coefficients of the ECG signal are computed with the wave. At block 512, the ends of the cross-correlation coefficients are truncated to provide a total pre-selected length (e.g., 856 sample points). At block 514, the absolute value of the cross-correlation coefficients are taken and scaled by a scaling factor (e.g., 700). If the standard deviation of the ECG is not less than a first pre-selected threshold (e.g., 0.5 mV) (block 516), then the FLATS output value is set to the number of coefficients below another pre-selected threshold (e.g., 0.375) (block 518). If the standard deviation is less than the first pre-selected threshold, then the coefficients are scaled (e.g., by 0.4 divided by the standard deviation) (block 520). At block 518, the FLATS value is set to the number of coefficients below the second pre-selected threshold. In further embodiments, other thresholds can be used. As discussed above, the FLATS value can be particularly useful in distinguishing a VF rhythm from asystole.

While several embodiments have been described above with reference to a particular type of interrogating wave (e.g., a Morlet wavelet), other interrogating waves can be used in further embodiments. For example, the waves used to interrogate the unknown rhythm epoch can be derived from wavelets such as the Mexican hat, Meyer, or other wavelet. In other embodiments, custom wavelets are used to interrogate signal. These custom waves may be tailored to be more specific for ECG classification schemes than the wavelets in general use. When choosing a particular wave which represents the ideal QRS complex or other cardiac ECG feature that a user is trying to identify, it is possible to pick out the parts of the unknown signal which resemble the interrogating wave. The peaks will be higher and a closer match when the signal contains QRS complexes or other features present in the wave. It is also possible to pick out an interrogating wave which more closely resembles the VF waveform to select out the signals with a high correlation with the VF waveform. In further embodiments, other specific cutoffs, amplitudes, and/or representative sections of the ECG waveform could be used in devising algorithms to separate ventricular fibrillation from organized rhythms and asystole. Still further, instead of a single interrogation wave, the use of a library of waveforms can be used to interrogate the signal. In this implementation it is possible to select multiple waveforms which are optimized in cross correlation with VF or with organized rhythms so that one can perform multiple analyses of the unknown signal.

### Examples

1. A method of identifying a cardiac rhythm in a person, the method comprising:
recording an electrocardiogram signal of the person; and
stratifying the signal, wherein a signal having a parameter value within a pre-determined range is categorized as a shockable signal and a signal having a parameter value outside the pre-determined range is categorized as a non-shockable signal.

2. The method of example 1 wherein recording an electrocardiogram signal comprises recording a signal having a length of approximately 3.8 seconds or less.

3. The method of example 1 wherein stratifying a signal having a parameter value within a pre-determined range comprises stratifying a signal having a parameter value within a statistically-determined range.

4. The method of example 1 wherein stratifying a signal having a parameter value comprises stratifying a signal having a value of at least one of standard deviation, fixed low-amplitude threshold sum, cross-correlation low-amplitude sum, and absolute peak amplitude.

5. The method of example 1, further comprising interrogating the signal with an interrogation wave.

6. The method of example 5 wherein interrogating the signal with an interrogation wave comprises interrogating the signal with a complex Morlet wavelet modified to include only a real component.

7. The method of example 5, further comprising generating cross-correlation coefficients by performing a cross-correlation of the signal and the interrogation wave.

8. The method of example 7, further comprising determining a count of cross-correlation coefficients having a magnitude below a pre-set fixed value.

9. The method of example 1, further comprising filtering the signal prior to stratifying the signal.

10. The method of example 9 wherein filtering the signal comprises band-pass filtering the signal.

11. The method of example 1, further comprising interrogating the signal with a plurality of waveforms.

12. The method of example 11 wherein interrogating the signal with a plurality of waveforms comprises interrogating the signal with a plurality of waveforms scaled in size based on a single original wave.

13. The method of example 12, further comprising:
generating cross-correlation coefficients by performing a cross-correlation of the signal and the interrogation waveforms;
squaring the cross-correlation coefficients;
calculating a mean of the squared cross-correlation coefficients; and
summing the number of squared cross-correlation coefficients that fall below a pre-selected percentage of the mean.

14. The method of example 1 wherein the method is performed while the person is undergoing cardiopulmonary resuscitation.

15. The method of example 1 wherein the method is performed while the person is undergoing cardiac monitoring.

16. A method of treating a patient undergoing cardiopulmonary resuscitation, the method comprising:
receiving an electrocardiogram signal of the patient;
interrogating the electrocardiogram signal with an interrogation wave;
determining a treatment for the patient based at least in part on the interrogating.

17. The method of example 16 wherein determining a treatment for the patient comprises identifying a cardiac rhythm of the patient as being ventricular fibrillation and indicating a treatment of defibrillatory shock.

18. The method of example 16, further comprising cross-correlating the electrocardiogram signal with the interrogation wave to generate cross-correlation coefficients.

19. The method of example 18, further comprising:
squaring the cross-correlation coefficients;
computing a mean of the squared cross-correlation coefficients; and
determining a count of squared cross-correlation coefficients below a pre-set percentage of the mean.

20. The method of example 18, further comprising determining a count of cross-correlation coefficients having a magnitude below a pre-set fixed value.

21. The method of example 16 wherein interrogating the electrocardiogram signal with an interrogation wave comprises interrogating the signal with a plurality of scaled waveforms.

22. The method of example 16 wherein determining a treatment for the patient comprises differentiating the patient's electrocardiogram signal from asystole and organized cardiac rhythm.

23. A physical computer-readable storage medium having stored thereon, computer-executable instructions that, if executed by a computing system, cause the computing system to perform operations comprising:
receiving a electrocardiogram signal;
interrogating the signal with an interrogation wave;
performing a cross-correlation of the signal and the interrogation wave;
calculating a parameter value based on the interrogating;
using a decision tree to stratify the signal based at least in part on the parameter value;
categorizing the signal as a shockable signal if the parameter value falls within a pre-determined range; and
categorizing the signal as a non-shockable signal if the parameter value falls outside the pre-determined range.

24. The physical computer-readable storage medium of example 23 wherein the operations further comprise distinguishing ventricular fibrillation from an organized rhythm or asystole.

25. The physical computer-readable storage medium of example 23 wherein the parameter comprises at least one of standard deviation, fixed low amplitude threshold sum, cross-correlation low amplitude sum, and absolute peak amplitude, and wherein the pre-determined range is statistically determined.

The systems and methods described herein offer several advantages over currently available methods of monitoring cardiac rhythms. For example, several of the methods described herein do not require that CPR be stopped for the analysis of the rhythm. The need to stop CPR in order to determine the state of the rhythm can result in an immediate and rapid deterioration in the condition of the myocardium. The methods of the present technology are not influenced adversely by the presence of chest compressions and ventilations performed with CPR. In further embodiments, the methods disclosed herein allow patients to be monitored in ambulances, helicopters and during movement when noise and artifacts can contaminate the ECG. This allows serious life threatening arrhythmias to be reliably identified so that alarms can be sounded and activated to alert personnel to the presence of a change in condition requiring immediate attention. It is widely recognized that in currently available monitoring devices, the alarms are routinely ignored because they are set off by artifacts such as patient movement so frequently as to make them unreliable indicators of the patient's condition. The method of the present technology provides for alarms that reliably signal the need for intervention without false alarms.

From the foregoing it will be appreciated that, although specific embodiments of the technology have been described herein for purposes of illustration, various modifications may be made without deviating from the spirit and scope of the technology. Further, certain aspects of the new technology described in the context of particular embodiments may be combined or eliminated in other embodiments. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A method of identifying a cardiac rhythm in a person, the method comprising:
recording an electrocardiogram signal of the person; and
stratifying the signal, wherein a signal having a parameter value within a pre-determined range is categorized as a shockable signal and a signal having a parameter value outside the pre-determined range is categorized as a non-shockable signal.

2. The method of claim 1 wherein recording an electrocardiogram signal comprises recording a signal having a length of approximately 3.8 seconds or less.

3. The method of claim 1 wherein stratifying a signal having a parameter value within a pre-determined range comprises stratifying a signal having a parameter value within a statistically-determined range.

4. The method of claim 1 wherein stratifying a signal having a parameter value comprises stratifying a signal having a value of at least one of standard deviation, fixed low-amplitude threshold sum, cross-correlation low-amplitude sum, and absolute peak amplitude.

5. The method of claim 1, further comprising interrogating the signal with an interrogation wave.

6. The method of claim 5 wherein interrogating the signal with an interrogation wave comprises interrogating the signal with a complex Morlet wavelet modified to include only a real component.

7. The method of claim 5, further comprising generating cross-correlation coefficients by performing a cross-correlation of the signal and the interrogation wave.

8. The method of claim 7, further comprising determining a count of cross-correlation coefficients having a magnitude below a pre-set fixed value.

9. The method of claim 1, further comprising filtering the signal prior to stratifying the signal.

10. The method of claim 9 wherein filtering the signal comprises band-pass filtering the signal.

11. The method of claim 1, further comprising interrogating the signal with a plurality of waveforms.

12. The method of claim 11 wherein interrogating the signal with a plurality of waveforms comprises interrogating the signal with a plurality of waveforms scaled in size based on a single original wave.

13. The method of claim 12, further comprising:
generating cross-correlation coefficients by performing a cross-correlation of the signal and the interrogation waveforms;
squaring the cross-correlation coefficients;
calculating a mean of the squared cross-correlation coefficients; and
summing the number of squared cross-correlation coefficients that fall below a pre-selected percentage of the mean.

14. The method of claim 1 wherein the method is performed while the person is undergoing cardiopulmonary resuscitation.

15. The method of claim 1 wherein the method is performed while the person is undergoing cardiac monitoring.

16. A method of treating a patient undergoing cardiopulmonary resuscitation, the method comprising:
receiving an electrocardiogram signal of the patient;
interrogating the electrocardiogram signal with an interrogation wave;
determining a treatment for the patient based at least in part on the interrogating.

17. The method of claim 16 wherein determining a treatment for the patient comprises identifying a cardiac rhythm of the patient as being ventricular fibrillation and indicating a treatment of defibrillatory shock.

18. The method of claim 16, further comprising cross-correlating the electrocardiogram signal with the interrogation wave to generate cross-correlation coefficients.

19. The method of claim 18, further comprising:
squaring the cross-correlation coefficients;
computing a mean of the squared cross-correlation coefficients; and
determining a count of squared cross-correlation coefficients below a pre-set percentage of the mean.

20. The method of claim 18, further comprising determining a count of cross-correlation coefficients having a magnitude below a pre-set fixed value.

21. The method of claim 16 wherein interrogating the electrocardiogram signal with an interrogation wave comprises interrogating the signal with a plurality of scaled waveforms.

22. The method of claim 16 wherein determining a treatment for the patient comprises differentiating the patient's electrocardiogram signal from asystole and organized cardiac rhythm.

23. A physical computer-readable storage medium having stored thereon, computer-executable instructions that, if executed by a computing system, cause the computing system to perform operations comprising:
receiving a electrocardiogram signal;
interrogating the signal with an interrogation wave;
performing a cross-correlation of the signal and the interrogation wave;
calculating a parameter value based on the interrogating;
using a decision tree to stratify the signal based at least in part on the parameter value;
categorizing the signal as a shockable signal if the parameter value falls within a pre-determined range; and
categorizing the signal as a non-shockable signal if the parameter value falls outside the pre-determined range.

24. The physical computer-readable storage medium of claim 23 wherein the operations further comprise distinguishing ventricular fibrillation from an organized rhythm or asystole.

25. The physical computer-readable storage medium of claim 23 wherein the parameter comprises at least one of standard deviation, fixed low amplitude threshold sum, cross-correlation low amplitude sum, and absolute peak amplitude, and wherein the pre-determined range is statistically determined.
